SEQUENCE LISTING (15 sequences)

SEQ ID NO: 1 - protein, 5 aa, Mus musculus
NYDIN

SEQ ID NO: 2 - protein, 11 aa, Mus musculus
WIFPGDGSTQY

SEQ ID NO: 3 - protein, 9 aa, Mus musculus
QTTATWFAY

SEQ ID NO: 4 - protein, 11 aa, Mus musculus
RASQSISDYLH

SEQ ID NO: 5 - protein, 7 aa, Mus musculus
YASQSIS

SEQ ID NO: 6 - protein, 9 aa, Mus musculus
QNGHSFPLT

SEQ ID NO: 7 - protein, 243 aa, Mus musculus
QVKLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTTVTVSSGGGGSGGGGSGGGGSDIELTQSPTTLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKQAA

SEQ ID NO: 8 - DNA, 731 bp, Mus musculus
caggtcaaactgcagcagtctggggctgaactggtaaagcctggggcttcagtgaaattgtcctgcaaggcttctggctacaccttcacaaactatgatataaactgggtgaggcagaggcctgaacagggacttgagtggattggatggatttttcctggagatggtagtactcaatacaatgagaagttcaagggcaaggccacactgactacagacacatcctccagcacagcctacatgcagctcagcaggctgacatctgaggactctgctgtctatttctgtgcaagacagactacggctacctggtttgcttactggggccaagggaccacggtcaccgtctcctcaggtggaggcggttcaggcggaggtggctctggcggtggcggatcggacatcgagctcactcagtctccaaccaccctgtctgtgactccaggagatagagtctctctttcctgcagggccagccagagtattagcgactacttacactggtaccaacaaaaatcacatgagtctccaaggcttctcatcaaatatgcttcccaatccatctctgggatcccctccaggttcagtggcagtggatcagggtcagatttcactctcagtatcaacagtgtggaacctgaagatgttggagtgtattactgtcaaaatggtcacagctttccgctcacgttcggtgctgggaccaagctggagctgaaacaggcggccgc

SEQ ID NO: 9 - DNA, 731 bp, Mus musculus
gtccagtttgacgtcgtcagaccccgacttgaccatttcggaccccgaagtcactttaacaggacgttccgaagaccgatgtggaagtgtttgatactatatttgacccactccgtctccggacttgtccctgaactcacctaacctacctaaaaaggacctctaccatcatgagttatgttactcttcaagttcccgttccggtgtgactgatgtctgtgtaggaggtcgtgtcggatgtacgtcgagtcgtccgactgtagactcctgagacgacagataaagacacgttctgtctgatgccgatggaccaaacgaatgaccccggttccctggtgccagtggcagaggagtccacctccgccaagtccgcctccaccgagaccgccaccgcctagcctgtagctcgagtgagtcagaggttggtgggacagacactgaggtcctctatctcagagagaaaggacgtcccggtcggtctcataatcgctgatgaatgtgaccatggttgtttttagtgtactcagaggttccgaagagtagtttatacgaagggttaggtagagaccctaggggaggtccaagtcaccgtcacctagtcccagtctaaagtgagagtcatagttgtcacaccttggacttctacaacctcacataatgacagttttaccagtgtcgaaaggcgagtgcaagccacgaccctggttcgacctcgactttgtccgccggcg

SEQ ID NO: 10 - DNA, 731 bp, Mus musculus
caggtcaaactgcagcagtctggggctgaactggtaaagcctggggcttcagtgaaattgtcctgcaaggcttctggctacaccttcacaaactatgatataaactgggtgaggcagaggcctgaacagggacttgagtggattggatggatttttcctggagatggtagtactcaatacaatgagaagttcaagggcaaggccacactgactacagacacatcctccagcacagcctacatgcagctcagcaggctgacatctgaggactctgctgtctatttctgtgcaagacagactacggctacctggtttgcttactggggccaagggaccacggtcaccgtctcctcagatggaggcggttcaggcggaggtggctctggcggtggcggatcggacatcgagctcactcagtctccaaccaccctgtctgtgactccaggagatagagtctctctttcctgcagggccagccagagtattagcgactacttacactggtaccaacaaaaatcacatgagtctccaaggcttctcatcaaatatgcttcccaatccatctctgggatcccctccaggttcagtggcagtggatcagggtcagatttcactctcagtatcaacagtgtggaacctgaagatgttggagtgtattactgtcaaaatggtcacagctttccgctcacgttcggtgctgggaccaagctggagctgaaacaggcggccgc

SEQ ID NO: 11 - protein, 243 aa, Mus musculus
QVKLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTTVTVSSDGGGSGGGGSGGGGSDIELTQSPTTLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKQAA

SEQ ID NO: 12 - protein, 243 aa, Artificial Sequence
  Mutated 8H9 scFv with decreased normal tissue adherence
QVKLQQSGAELVEPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTTVTVSSDGGGSGGGGSGGGGSDIELTQSPTTLSVTPGDQVSLSCRASQSISDYLHWYQQKSHESPQLLIKYASQSISGIPSRFSGSGSGSDFTLSINSVEPEDVGVYYCQNGHSFPLTFGAGTELELEQAA

SEQ ID NO: 13 - protein, 18 aa, Mus musculus
NPVLQQDAHSSVTITPQR

SEQ ID NO: 14 - protein, 26 aa, Mus musculus
SPTGAVEVQVPEDPVVALVGTDATLR

SEQ ID NO: 15 - protein, 534 aa, Mus musculus
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

(19) United States
(12) Patent Application Publication
(10) Pub. No.: US 2010/0143245 A1
(43) Pub. Date: Jun. 10, 2010

(54) USES OF MONOCLONAL ANTIBODY 8H9

(75) Inventor: Nai-Kong V. Cheung, Purchase, NY (US)

Correspondence Address: LAW OFFICES OF ALBERT WAI-KIT CHAN, PLLC, 141-07 20TH AVENUE, WORLD PLAZA, SUITE 604, WHITESTONE, NY, 11357, US

(73) Assignee: SLOAN-KETTERING INSTITUTE FOR CANCER RESEARCH, New York, NY (US)

(21) Appl. No.: 12/531,828

(22) PCT Filed: Mar. 24, 2008
(86) PCT No.: PCT/US08/58030

§ 371 Date: Sep. 17, 2009

Related U.S. Application Data
(60) Provisional application No. 60/896,416, filed on Mar. 22, 2007.
(60) Provisional application No. 60/915,672, filed on May 2, 2007.

(51) Int. Cl. A61K 51/10; A61K 39/395; C07K 16/00; G01N 33/566; A61P 35/00

(52) U.S. Cl. 424/1.49; 424/174.1; 424/133.1; 424/134.1; 530/387.1; 436/501

(57) ABSTRACT

The present invention discloses monoclonal antibody 8H9 which binds to the 4Ig domain isoform of the human B7-homolog 3, 4Ig-B7H3. The present invention provides a method of improving the prognosis or prolonging the survival of a subject bearing tumor cells, the method comprises administering to the subject a composition comprising an effective amount of an agent capable of binding to an antigen recognized by monoclonal antibody 8H9.

This application claims the benefit of priority of U.S. Ser. No. 60/896,416, filed Mar. 22, 2007, and U.S. Ser. No. 60/915,672, filed May 2, 2007. The entire contents and disclosures of the preceding applications are incorporated by reference into this application.

### FIELD OF THE INVENTION

This invention relates to uses of monoclonal antibody 8H9 or derivates thereof in treating cancer patients.

### BACKGROUND OF THE INVENTION

Tumor-restricted surface antigens may be targets for diagnosis and immune-based therapies. An ideal tumor antigen for targeted immunotherapy should be absent on normal tissues and abundantly expressed on tumor cell surface. Moreover, a “generic” tumor-specific antigen expressed on tumor cells of varying lineage recognized by monoclonal antibodies may have broader utility in antibody-based strategies. A novel 58 kD surface tumor-associated antigen recognized by a murine monoclonal antibody 8H9 has been reported previously (see e.g. U.S. patent application publication US 2005/0169932). The antigen recognized by 8H9 was expressed on cell membranes of a broad spectrum of tumors of neuroectodermal, mesenchymal and epithelial origin, with restricted distribution on normal tissues. This novel antibody-antigen system is very promising for tumor targeting and immunotherapy.

Monoclonal antibody 8H9 can be used for tumor targeting and imaging, and purging of tumor cells. The 8H9 antigen is also a potential target for antibody-based immunotherapy against a broad spectrum of human cancers, including neuroblastoma, brain tumors, desmoplastic small round cell tumor, rhabdomyosarcoma, osteosarcoma, Ewings sarcoma, PNET, melanoma, sarcoma, wilm's tumor, hepatoblastoma, and carcinomas of various tissue origins. Construction of 8119 single chain antibody and antibody-fusion constructs have also been described (see e.g. U.S. patent application publication US 2005/0169932).

The present disclosure provides further data on using monoclonal antibody 8H9 to improve the prognosis and/or prolong the survival of a subject bearing tumor cells.

Throughout this application, various references are cited. Disclosures of these publications in their entireties are hereby incorporated by reference into this application to more fully describe the state of the art to which this invention pertains.

### SUMMARY OF THE INVENTION

The present invention provides a method of improving the prognosis or prolonging the survival of a subject bearing a tumor, comprising administering to the subject a composition comprising an effective amount of an agent capable of binding to an antigen recognized by mononclonal antibody 8H9.

The present invention also provides a method of improving the prognosis or prolonging the survival of a subject bearing a tumor expressing an antigen recognized by monoclonal antibody 8H9. This method comprises administering to the subject a composition comprising an effective amount of an agent capable of binding to an antigen recognized by mononclonal antibody 8H9.

The present invention also provides a method of screening for antibodies that have the same or similar binding specificity as monoclonal antibody 8H9, comprising the step of contacting candidate antibodies with a polypeptide comprising the sequence of SEQ ID NO.15, or a fragment thereof, wherein antibodies that bind to the polypeptide are antibodies that have the same or similar binding specificity as monoclonal antibody 8H9. The present invention also provides an antibody identified by the above screening method.

The present invention also provides an antigen which is recognized by monoclonal antibody 8H9, wherein the antigen has at least about 10%, preferably between 10% and 99% homology to SEQ ID NO.15.

### DETAILED DESCRIPTION OF THE FIGURES

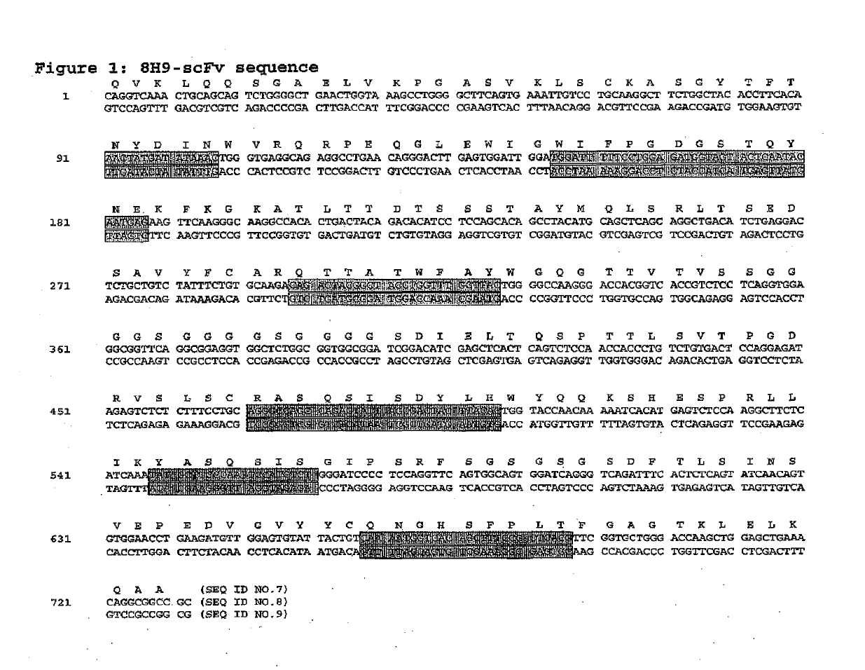

FIG. 1 shows 8H9 scFv amino acid sequence (SEQ ID NO.7) and gene sequences (sense and complementary, SEQ ID NOs. 8-9). Complementary determining regions (CDR) are marked in boxes in the following order: CDR-1 (HC, heavy chain), CDR-2 (HC), CDR-3 (HC), CDR-1 (LC, light chain), CDR-2 (LC), CDR-3 (LC).

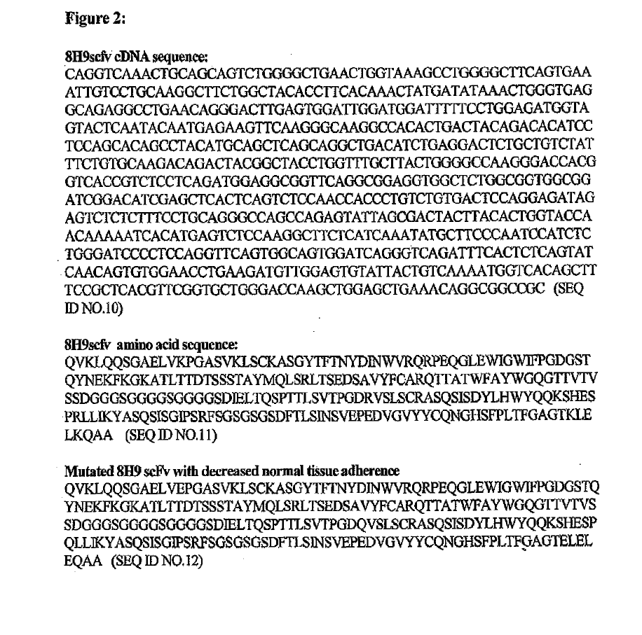

FIG. 2 shows nucleotide and amino acid sequences of 8H9scFv (SEQ ID NOs.10-12). Mutated 8H9 scFv carries the following site-directed mutagenesis (VH: K13E and VL: R18Q, R45Q, K103E, K107E) to decrease PI from 6.4 to 4.8, and net charge from −1 to −9, a strategy to decrease nonspecific normal tissue adherence.

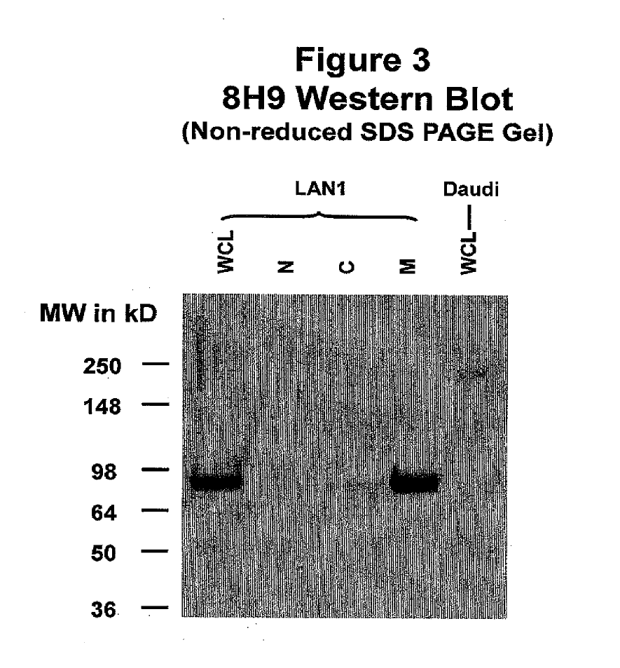

FIG. 3 shows non-reduced SDS-PAGE of 8H9 Western Blot.

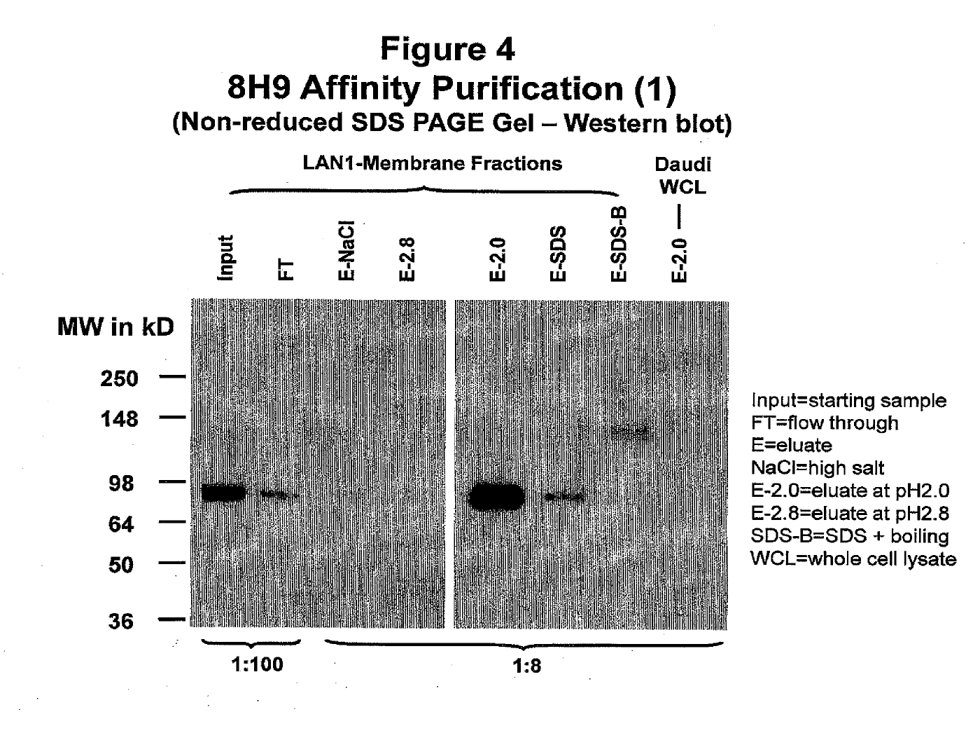

FIG. 4 shows 8H9 affinity purification (non-reduced SDS-PAGE, Western Blot).

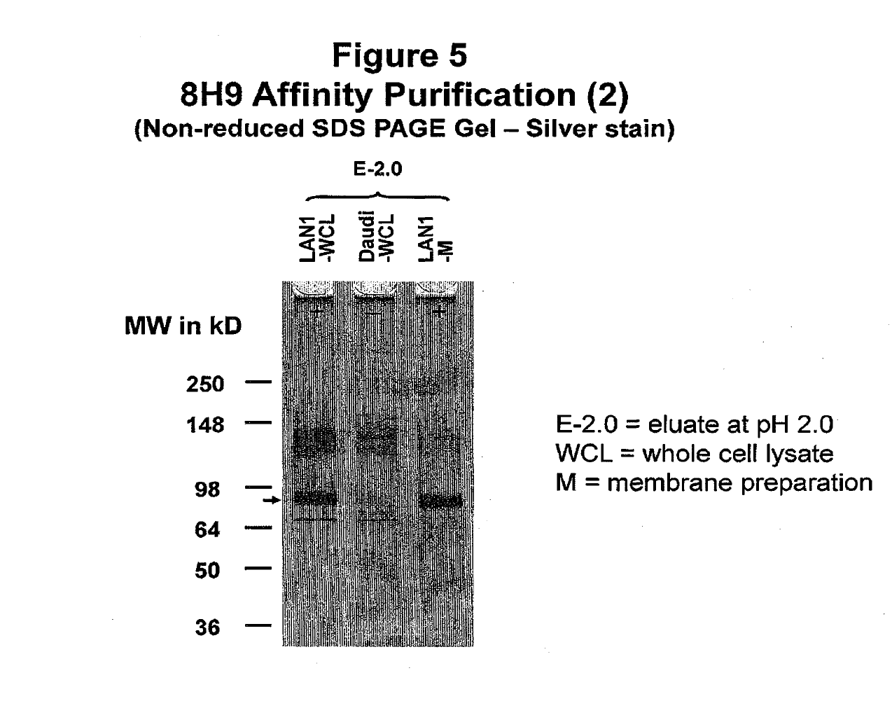

FIG. 5 shows 8H9 affinity purification (non-reduced SDS-PAGE, silver stain).

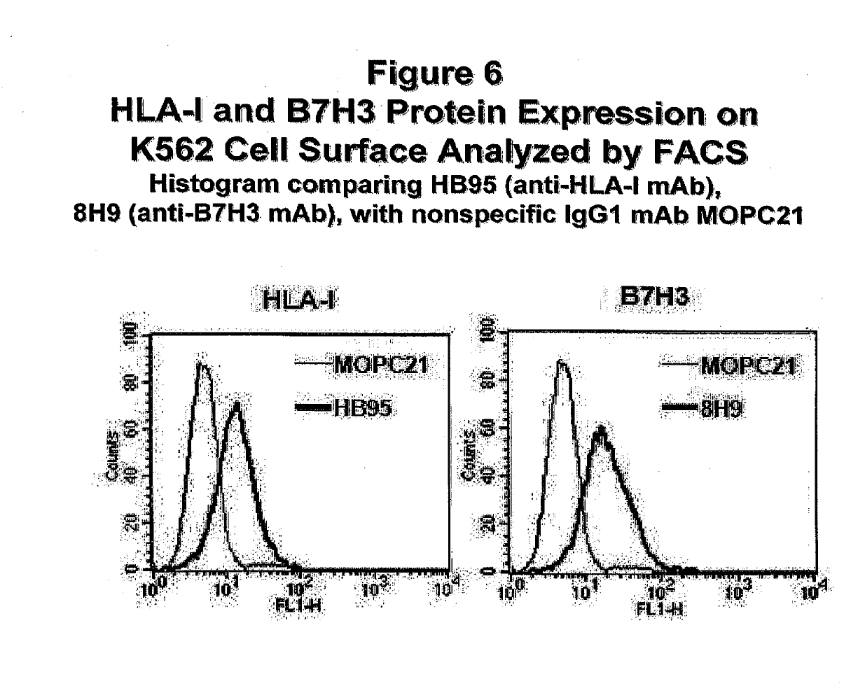

FIG. 6 shows HLA-I (MHC class I) and B7H3 protein expression on K562 cell surface analyzed by FACS.

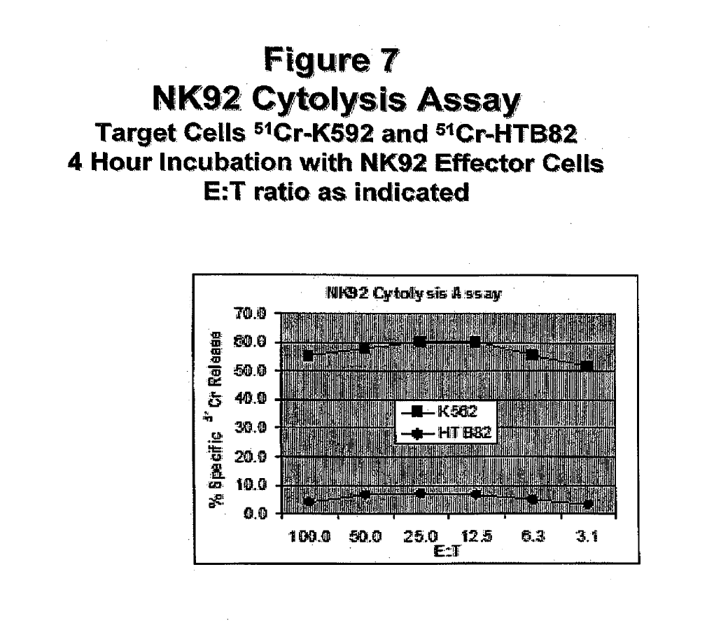

FIG. 7 shows the cytolytic activity of NK92 cells against K562 and HTB82 cells (results of Chromium release assay for cell-mediated cytolysis).

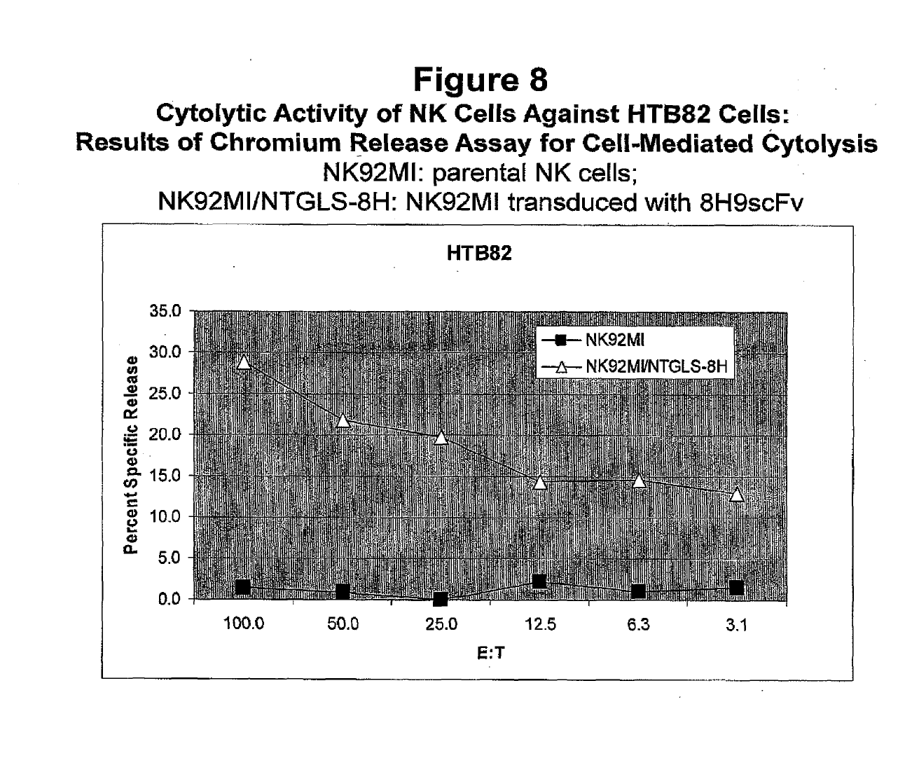

FIG. 8 shows the cytolytic activity of NK cells against HTB82 cells (results of Chromium release assay for cell-mediated cytolysis). NK92MI: parental NK cells; NK92MI/NTGLS-8H: NK92MI transduced with 8H9scFv.

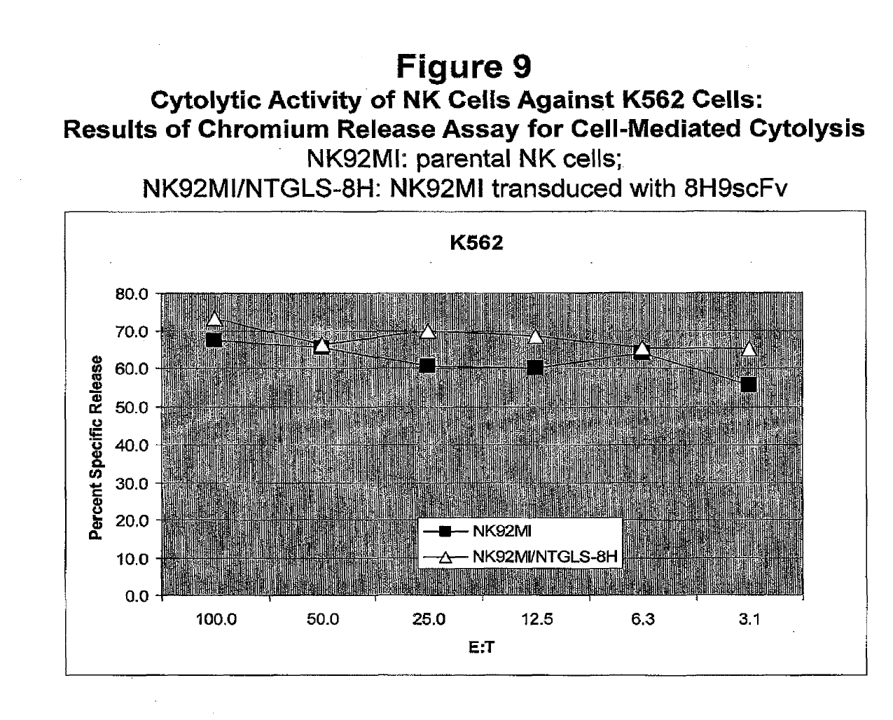

FIG. 9 shows the cytolytic activity of NK cells against K562 cells (results of Chromium release assay for cell-mediated cytolysis). NK92MI: parental NK cells; NK92MI/NTGLS-8H: NK92MI transduced with 8H9scFv.

### DETAILED DESCRIPTION OF THE INVENTION

The present invention provides a method of improving the prognosis or prolonging the survival of a subject bearing a tumor, comprising administering to the subject a composition comprising an effective amount of an agent capable of binding to an antigen recognized by mononclonal antibody 8H9. As used herein, “improving the prognosis” refers to early detection of cancer and early initiation of treatment that would lead to a future course of disease with possible recovery or cure of the disease, whereas “prolonging the survival” refers to increasing the life expectancy after the diagnosis of cancer. In one embodiment, the tumor expresses an antigen recognized by mononclonal antibody 8H9.

In one embodiment, the antigen recognized by mononclonal antibody 8H9 is a polypeptide comprising the sequence of SEQ ID NO.15. In another embodiment, the antigen is a polypeptide homolog of SEQ ID NO.15. In general, there is at least about 10% homology, or at least about 15% homology, or at least about 25% homology, or at least about 35% homology, or at least about 45% homology, or at least about 55% homology, or up to 100% homology to SEQ ID NO.15. One of ordinary skill in the art would readily a homolog or ortholog of SEQ ID NO.15 (see e.g. Table 1).

TABLE 1
Orthologs of CD276

| | | | Human | |
|---|---|---|---|---|
| Organism | Gene | Description | Similarity | NCBI accessions |
| dog | CD276¹ | CD276 | 90.65(n) | 487638 XM_849111.1 XP_854204.1 |
| (Canis | | molecule | 93.97(a) | |
| familiaris) | | | | |
| chimpanzee | LOC467818¹ | CD276 | 99(n) | 467818 XM_523213.2 XP_523213.2 |
| (Pan | | molecule | 98.88(a) | |
| troglodytes) | | | | |
| rat | Cd276¹ | CD276 antigen | 89.35(n) | 315716 NM_182824.2 NP_877976.1 |
| (Rattus | | | 93.47(a) | |
| norvegicus) | | | | |
| mouse | Cd2764 | CD276 | 88.89(n)¹ | 102657¹ NM_133983.3¹ NP_598744.1¹ |
| (Mus | Cd276¹ | antigen^{1,4} | 92.78(a)¹ | AI415625⁴ AI593640⁴ (see all 16) |
| musculus) | | | | |
| chicken | CD276¹ | CD276 | 73.36(n) | 415315 XM_413702.2 XP_413702.2 |
| (Gallus | | molecule | 68.51(a) | |
| gallus) | | | | |
| zebrafish | LOC572193¹ | similar to | 62.37(n) | 572193 XM_695881.2 XP_700973.2 |
| (Danio rerio) | | CD276 antigen | 55.68(a) | |
| African | XI.15387^{1~} | Xenopus laevis | 68.63(n) | CB207657.1 |
| clawed frog | | transcribed | | |
| (Xenopus | | sequence with | | |
| laevis) | | weak similarity | | |
| | | more | | |

Human similarity showing % homology at the nucleotide level (n) or amino acid level (a).

In one embodiment, the agent in the above composition is a polypeptide comprising Complementary Determining Regions (CDRs) derived from monoclonal antibody 8H9. Examples of such polypeptide include, but are not limited to, single chain antibody or antibody-fusion construct. As used herein, “single chain antibody” refers to reduction of an immunoglobulin molecule (4 peptide chains) into a single peptide that retains immunoreactivity and specificity for the antigen or for the tumor, usually in the form of a single peptide incorporating the heavy chain and the light chain of the immunoglobulin, whereas “antibody-fusion construct” refers to chemically or genetically linking such single chain antibody to another protein or peptide to form a novel antibody-fusion construct.

In one embodiment, such polypeptide comprises CDRs of SEQ ID NOs.1-3, 4-6, or 1-6. Preferably, sequences other than the CDRs on the above polypeptides are of human origin. In another embodiment, the polypeptide has an amino acid sequence of SEQ ID NO. 7 or 12. Moreover, the agent in the above composition can be directly or indirectly coupled to a labeling agent or a cytotoxic agent. Representative examples of such labeling agent or cytotoxic agent include, but are not limited to, radioisotopes and toxins such as pseudomonas exotoxin.

In general, the above composition can be administered intraperitoneally, intravenously, intrathecally, by Ommaya reservoir or by spinal tap, intraparenchymally into the tumors (either primary or metastatic), or into tissues surrounding the tumor.

The agent of the above compositions, when labeled with a radioisotope, may be used for both therapeutic purposes and for imaging purposes. In one embodiment, such agent in the above composition is administered at 0.01 mg to 20 mg per injection, carrying 1 mCi to 100 mCi of 131-Iodine, and in a preferred embodiment is used therapeutically.

In another embodiment, the agent in the above composition is administered at 0.01 mg to 20 mg per injection, carrying 1 mCi to 100 mCi of 124-Iodine, and in a preferred embodiment is used for imaging and dosimetry purposes.

In another embodiment, the agent in the above composition is administered at 0.01 mg to 20 mg per injection, carrying biologically equivalent radioactive doses of beta-emitters or alpha emitters to 1 mCi to 100 mCi of 131-Iodine, wherein such beta-emitters or alpha emitters may be 213-Bismuth, 212-Bismuth, 111-Indium, 118-Rhenium, 90-Yttrium, 225-Actinium, and 177-Lutetium, or 85-Astatine.

In another embodiment, the agent in the above composition is administered at 0.01 mg to 20 mg per injection, carrying biologically equivalent radioactive doses of positron-emitters to 1 mCi to 100 mCi of 124-Iodine, wherein such positron-emitters may be 94m-Technetium, 64-Copper, 89-Zirconium, 68-Gallium, 66-Gallium, 76-Bromium, 86-Yttrium, 82-Rubidium, 110m-Indium, 13-Nitrogen, 11-Carbon or 18-Fluorine.

In a preferred embodiment, the above composition is administered after the subject has been treated with one or more other cancer treatments. In a further embodiment, the above composition, is administered simultaneously or sequentially while the subject is being treated with one or more other cancer treatments. Examples of such other cancer treatments include, but are not limited to, surgery, chemotherapy, and radiation.

The present invention also provides use of an agent, which has the characteristics described above (e.g. capable of binding to an antigen recognized by monoclonal antibody 8H9) as a medicament for improving the prognosis or prolonging the survival of a subject bearing a tumor. In one embodiment, the tumor expresses an antigen recognized by mononclonal antibody 8H9. The routes and doses of administrating a composition comprising the agent can be readily determined by one of ordinary skill in the art. For example, the composition can be administered according to the doses and routes of administration described above.

The present invention also provides a method of screening for antibodies that have the same or similar binding specificity as monoclonal antibody 8H9, comprising the step of contacting candidate antibodies with a polypeptide comprising the sequence of SEQ ID NO.15, or a fragment thereof, wherein antibodies that bind to the polypeptide are antibodies that have the same or similar binding specificity as monoclonal antibody 8H9. The present invention also comprises an antibody identified by the method described herein.

The present invention also provides an antigen which is recognized by monoclonal antibody 8H9, wherein the antigen has at least about 10, preferably between 10% and 99% homology to SEQ ID NO.15.

The present invention also provides a method of upregulating anti-metastatic immune response in NK/T cells comprising the steps of blocking B7H3 receptors present on NK/T cells with an appropriate agent.

This invention also provides methods for screening agents which competitively inhibit the binding of monoclonal antibody 8H9 to its target, comprising steps of contacting candidate with the target in conditions permitting the binding of the candidate and the target. In a preferred embodiment, the above method further comprises detection of formation of a complex and the candidate and the target. In this embodiment, the target is B7H3, also known as CD276, and the agent may be an antibody, a peptide, a cell surface protein, or a ligand.

The invention will be better understood by reference to the Experimental Details which follow, but those skilled in the art will readily appreciate that the specific experiments detailed are only illustrative, and are not meant to limit the invention as described herein, which is defined by the claims which follow thereafter.

### Example 1

### Improved Outcome with Combined Modality Including 131-Iodine-8H9 Radioimmunotherapy Delivered through the Cerebrospinal Fluid

Background: Primary brain tumors and cancers metastatic to the CNS (brain parenchyma or leptomeninges [LM]) are difficult to control. Antibody-based targeted therapies administered through the cerebrospinal fluid (CSF) compartment have therapeutic potentials. Monoclonal antibody 8H9 is a murine IgG1 antibody reactive with a wide spectrum of human solid tumors. 131-Iodine-8H9 administered through the Ommaya has favorable pharmacokinetics in non-human primates with minimal toxicities.

Methods: In a phase I study, 15 patients (pts, ages 2-34 years) (1 melanoma, 3 recurrent ependymoma, 8 relapsed CNS neuroblastoma [NB], 3 recurrent medulloblastoma) received 2 mCi intra Ommaya 131I-8H9 for dosimetry followed 1 week later by an intra-Ommaya treatment dose of 10 (n=3 pts), 20 (n=3), 30 (n=6), or 40 (n=3) mCi. Serial cerebrospinal fluid (CSF) and blood were sampled for dosimetry calculations. Nuclear scans were performed at 24 hours post to study 131I-8H9 localization. The 131-Iodine-8H9 dosimetry and treatment doses were repeated after 1 month if the pt had no PD.

Results: Side effects included grade 1 or 2 fever, headache or vomiting; one had a transient grade 3 ALT elevation on first injection (30 mCi). Calculated mean radiation dose to the CSF was 35.7 (range 15-79) cGy/mCi; mean blood dose was 2.4 cGy/mCi. Of the 15 pt, 8 (group #1) had primary diagnosis of neuroblastoma. When they developed CNS metastasis (at median age of 3.8 years) they were treated with a salvage regimen which included 131-Iodine-8H9. All 8 pts remain alive progression-free (3+, 10+, 16+, 16+, 18+, 18+, 20+, 30+months since 131-Iodine-8H9, and 5-43+ months since CNS/LM relapse); in one pt, 131-Iodine-8H9 achieved CR of LM disease. In contrast, median time to death from the onset of CNS/LM NB was 5.4 months for 27 historical controls. Acute side effects were self-limited; at 40 mCi dose no DLT was seen.

Conclusion: Similar to CNS metastases in most other solid tumors, conventional therapies have been ineffective for NB-CNS. Intra-Ommaya 131-Iodine-8H9 (1) is safe, (2) has favorable dosimetry to CSF and marrow, and (3) may have clinical utility when added to salvage therapy using conventional modalities in the treatment of 8H9-positive LM/CNS cancers.

### Example 2

### Improved Resolution and Contrast Imaging of CNS Tumors Using 124-Iodine-8H9 in PET/CT Scans

Background: As stated in Example 1, antibody-based targeted therapies administered through the cerebrospinal fluid (CSF) compartment have therapeutic potential, and radiolabeled 131-Iodine-8H9 can be used to treat metastatic disease. A patient's prognosis will be improved not only with improved treatment but also with improved detection of the metastatic disease and improved dosimetry. The following example describes an improved means of detection of neuroblastoma.

Methods: Five patients were injected intrathecally with 124-Iodine-8H9 and serial PET/CT imaging and CSF sample was performed. Patients had CNS tumors (choroid plexus carcinoma, metastatic rhabdomyosarcoma, and metastatic neuroblastoma. 1.7-2 mCi 124-Iodine-8H9 was administered through an Ommaya reservoir. PET/CT scans were obtained at approximately 4, 24, and 48 hours post-injection. Serial cerebrospinal fluid (CSF) samples through 48 hours were obtained. Images were analyzed by placing regions of interest on the spinal column at all three time points. PET images provided direct activity measurements within the CSF compartment.

Results: 124-Iodine-8H9 PET scans provided high resolution images of the antibody distribution with targeting of disease in the 2 patients with structural lesions on MRI. At 24 hours, most of the antibody had cleared from the ventricles and had distribution through the thecal sac and around the cerebral convexities. The distribution corresponded well with pre-treatment ¹¹¹in-DTPA cisternography. Systemic activity in liver, spleen and bladder was seen at 24 and 48 hours. The biological T1/2 clearance ranged from 8.9 hours to 64.6 hours with corresponding doses of 14.1 ti 92.9 cGy/mCi to CSF.

Conclusion: 124-Iodine-8H9 PCT/CT provides higher resolution and contrast images than SPECT with 131-Iodine-8H9 for distribution, targeting and dosimetry.

### Example 3

### 8H9 Antibody Recognizes the 4Ig Domain Isoform of the Human B7-Homolog 3, 4Ig-B7H3

The following example describes biochemical characterization of the antigen recognized by the 8H9 antibody. The identity of the antigen is the 4Ig domain isoform of the human B7-homolog 3, 4Ig-B7H3.

Cell Culture. The human neuroblastoma cell line LAN-1 was provided by Dr. Robert Seeger (Children's Hospital of Los Angeles, Los Angeles, Calif.). Human rhabdomyosarcoma cell line HTB82, osteosarcoma cell line U2OS, and Burkitt's lymphoma cell line Daudi were purchased from American Type Culture Collection (Bethesda, Md.). All cell lines were grown in RPMI 1640 medium supplemented with 10% bovine calf serum, 2 mM glutamine, 100 U/ml penicillin, and 100 μg/ml streptomycin at 37° C. in a 5% CO2 incubator.

Monoclonal Antibodies. Both 8H9 and control MoAb 5F9 are murine IgG1 and were produced against human neuroblastoma. They were purified by protein A (GE Healthcare, Piscataway, N.J.) affinity chromatography before use.

Whole Cell Lysates and Western Blot. 8H9-positive cell lines (LAN-1, HTB82 and U2OS) and 8H9-negative cell line (Daudi) were grown to ˜80% confluence. Cells were harvested using 2 mM EDTA and washed with ice-cold PBS.

Native PAGE was performed using NativePAGE Novex Bis-Tris Gel System (Invitrogen, Carlsbad, Calif.) according to the manufacturer's instructions. Briefly, cells were lysed on ice (20 min) in NativePAGE 1× Sample Buffer plus 1% detergent (either Triton-X100 or n-dodecyl-β-D-maltoside (DDM)) and protease inhibitor cocktail tablets (Roche Applied Science, Germany). The lysates were clarified by centrifugation at 14,000 rpm for 20 min at 4° C. 50 μg whole cell lysates were analyzed by NativePAGE Novex 4-16% Bis-Tris Gels.

SDS-PAGE under nonreducing or reducing conditions was performed using Tris-Glycine Ready Gel System (Bio-Rad, Hercules, Calif.). Briefly, cells were lysed on ice (20 min) in Triton Lysis Buffer (50 mM Tris-HC1, pH 7.2, 50 mM NaCl, 10% glycerol, 1% Triton X-100, and protease inhibitor cocktail tablets). The lysates were clarified as above. 25˜50 pg whole cell lysates were analyzed by 4-15% Tris-HCl Gels.

After electrophoresis in either PAGE, samples were transferred onto Immun-Blot PVDF Membrane (Bio-Rad), blocked for one hour at room temperature (RT) with 10% dry milk in TEST, and incubated with primary antibodies (8H9 at 10-20 μg/ml, 5F9 at 20 μg/ml) for 3 hrs at RT. The membrane was then washed with TBST, and incubated with secondary Peroxidase-conjugated AffiniPure Goat Anti-Mouse IgG (H+L) (Jackson ImmunoResearch, West Grove, Pa.). Bands were detected with SuperSignal West Pico Chemiluminescent Substrate (PIERCE, Rockford, Ill.).

Subcellular Fractionation. For crude membrane preparation, LAN-1 cells were pipetted off the tissue culture dish, washed with ice-cold PBS, and lysed on ice in sucrose buffer (0.25 M sucrose, 5 mM Tris-HC1, pH 7.2, and protease inhibitor cocktail tablets) with a bounce homogenizer (Kontes, Vineland, N.J.). Centrifugation for 10 min at 1000 g pelleted all nuclei, as judged microscopically. The 1000 g supernatant was ultracentrifuged at 100,000 g for 30 min in a Beckman L-70K (25,000 rpm, SW41Ti rotor) to give membrane particulate (P100) and cytosolic (S100) fractions. Cytosolic fraction was adjusted to 1% Triton, while crude nuclear and membrane fractions were resuspended in Triton Lysis Buffer and clarified before use.

8H9 Antigen Affinity Purification. 8H9 antigen was purified from LAN-1 cell extracts by immuno-affinity chromatography using MoAb 8H9. The 8H9 affinity column was prepared using Pierce's Protein G IgG Plus Orientation Kit (PIERCE, Rockford, Ill.) according to the manufacturer's instructions.

Four mg of LAN-1 whole cell lysates or equivalent membrane fraction prepared as above were incubated overnight at 4° C. with 20 μl 8H9-protein G Sepharose (covalently crosslinked with disuccinimidyl suberate (DSS), 3 mg bound 8H9/ml beads). After extensive washing with Triton Lysis Buffer, the column was eluted sequentially with 50 mM Tris-HCl, pH 7.2 containing 1M NaCl, 0.1 M Glycine-HCl, pH 2.8 and pH 2.0, SDS Sample Buffer (62.5 mM Tris-HCl, pH 6.8, 2% SDS, 10% glycerol, 0.005% Bromophenol blue), and SDS Sample Buffer plus boiling in water for 5 min. A small aliquot of eluates was monitored for the presence of 8H9 antigen by Western Blot analysis under nonreducing conditions using 8H9 antibody. One-fourth of the eluates was also analyzed by silver staining (SilverQuest Silver Staining Kit, Invitrogen). Finally, one-half of the 8H9 antigen-positive eluate (0.1 M Glycine-HCl, pH 2.0 eluted fraction) was analyzed by colloidal Coomassie blue staining (GelCode Blue Stain Reagent, PIERCE), and the 8H9 antigen-positive band was sent for mass spectrometric identification by MSKCC Microchemistry and Proteomics Core Facility.

### Results

Western Blot Detection of 8H9 Antigen. 8H9 antigen was first detected by 8H9 MoAb under native conditions using NativePAGE Novex Bis-Tris Gel system. A single band was detected in all 8H9-positve cell lines (LAN-1, HTB82 and U2OS) but not 8H9-negative cell line (Daudi), as defined by flow cytometry analysis, using 1% nonionic detergent (either Triton-X100 or DDM) (data not shown). The detection was specific since 5F9, a control MoAb against Ku70 protein, detected a band with a different size (data not shown).

Later, 8H9 antigen was also detected by 8H9 MoAb under nonreducing conditions using Tris-Glycine Ready Gel SDS-PAGE system. Just like under native conditions, a single band (˜85 KD using Invitrogen SeeBlue Plus2 Pre-Stained Standard as protein molecular weight marker) was detected in all 8H9-positve cell lines (LAN-1, HTB82 and U20S) but not 8H9-negative cell line (Daudi), using 1% Triton Lysis Buffer (FIG. 3, and data not shown). The detection was specific, since 5F9 (an IgG1 specific for Ku70) did not detect a band at the same size (data not shown). The size of 8H9 antigen detected is consistent with previous data using 8H9 radio-immunoprecipitation. We were unable to detect 8H9 antigen by Western Blot analysis under reducing conditions (data not shown), suggesting 8H9 recognize a conformational sensitive epitope.

After subcellular fractionation, 8H9 antigen was detected predominantly in membrane fraction (FIG. 3), which is consistent with previous data that 8H9 antigen is a cell surface antigen. Enrichment of 8H9 antigen in the membrane fraction was then undertaken using affinity purification.

Affinity Purification of the 8H9 Antigen. LAN-1 cell line was selected for antigen purification because its relatively high level expression of 8H9 antigen and ability of growing rapidly in tissue culture. 8H9 affinity column was prepared by covalently conjugating Fc portion of 8H9 to protein G of the gel matrix in a defined orientation, allowing exposure of a higher number of free antibody binding sites for antigen binding. Utilizing the NHS-ester DSS in place of the traditional imidoester DMP for crosslinking also significantly prevents the leaching of antibody from the support.

After incubating either LAN-1 (and Daudi as negative control) whole cell lysates or LAN-1 membrane fraction with 8H9-protein G Sepharose overnight, a significant portion (>50%) of 8H9 antigen was bound to the Sepharose (FIG. 4, and data not shown). 8H9 antigen was eluted specifically and predominantly in 0.1 M Glycine-HCl, pH 2.0 as monitored by Western Blot analysis (FIG. 4, and data not shown), suggesting a very strong interaction between 8H9 antibody and its antigen. After silver staining the same eluate, a clear band was detected accordingly only in LAN-1 cell extracts but not in Daudi cell extracts (FIG. 5). The eluate was also clean enough in the 85 KD vicinity for mass spec analysis. Finally, enough quantity of 8H9 antigen in the band (˜10 ng, visible with colloidal Coomassie staining, data not shown) was collected and sent for mass spectrometric identification.

Mass spectrometric identification Tryptic digests were subjected to a micro-clean-up procedure using 2 μL bed-volume of Poros 50 R2 (PerSeptive) reversed-phase beads, packed in an Eppendorf gel-loading tip. Mass spectrometry (MALDI-ReTOF) was performed on peptide pools (16 & 30% MeCN) recovered from the RP-microtip column using a Bruker Ultraflex TOF/TOF instrument with delayed extraction. For mass fingerprinting, experimental masses (m/z) combined from both MALDI-ReTOF experiments were used to search a non-redundant human protein database (NR; ˜192,489 entries; NCBI; Bethesda, Md.), using the PeptideSearch (Matthias Mann, Max-Planck Institute for Biochemistry, Martinsried, Germany) algorithm. A molecular weight range twice the predicted weight was covered, with a mass accuracy restriction better than 50 ppm, and maximum one missed cleavage site allowed per peptide. Mass spectrometric sequencing (MALDI-TOF-MS/MS) of selected peptides from partially fractionated pools was done on a Bruker Ultraflex TOF/TOF instrument in ‘LIFT’ mode, and the fragment ion spectra taken to search human databases using the MASCOT MS/MS Ion Search program (Matrix Science). Two peptide sequences from the peptide digest were identified: NPVIQQDAHSSVTITPQR (SEQ ID NO.13), and SPTGAVEVQVPEDPVVALVGTDATLR (SEQ ID NO.14).

These yielded an unequivocal identification of the antigen as 4Ig-B7H3, the 4Ig domain isoform of the human B7-homolog 3, also named CD276, accession number of NM_{—}001024736.1, which codes for a peptide of 534 amino acids, of 57235 kD molecular weight. The gene is located on chromosome 15824.1. The amino acid sequence of the mature human protein is as follows (with the potential N-glycosylation sites underlined):

(SEQ ID NO. 15)

MLRRRGSPGMGVHVGAALGALWFCLTGALEVQVPEDPVVALVGTDATLCC
SFSPEPGFSLAQLNLIWQLTDTKQLVHSFAEGQDQGSAYANRTALFPDLL
AQGNASLRLQRVRVADEGSFTCFVSIRDFGSAAVSLQVAAPYSKPSMTLE
PNKDLRPGDTVTITCSSYQGYPEAEVFWQDGQGVPLTGNVTTSQMANEQG
LFDVHSILRVVLGANGTYSCLVRNPVLQQDAHSSVTITPQRSPTGAVEVQ
VPEDPVVALVGTDATLRCSFSPEPGFSLAQLNLIWQLTDTKQLVHSFTEG
RDQGSAYANRTALFPDLLAQGNASLRLQRVRVADEGSFTCFVSIRDFGSA
AVSLQVAAPYSKPSMTLEPNKDLRPGDTVTITCSSYRGYPEAEVFWQDGQ
GVPLTGNVTTSQMANEQGLFDVHSVLRVVLGANGTYSCLVRNPVLQQDAH
GSVTITGQPMTFPPEALWVTVGLSVCLIALLVALAFVCWRKIKQSCEEEN
AGAEDQDGEGEGSKTALQPLKHSDSKEDDGQEIA

TABLE 2

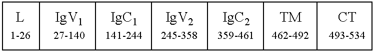

| B7 Member | Action On Immunity | Receptor |
|---|---|---|
| B7H1 (CD274) | inhibitory | PD-1 (CD279) |
| B7DC (CD273) | stimulatory/inhibitory | PD-1 (CD279) |
| B7H2 (LICOS) | TH2 skewing | ICOS |
| B7H3 (CD276) | stimulatory/inhibitory | ? ? |
| B7H4 (B7X) | inhibitory | ? ? |

To date, the new B7s include B7H1, B7DC, B7H2, B7H3, and B7H4 (see Table 2).⁴ Although their mRNAs are fairly ubiquitously, these protein molecules may be differentially regulated at the post-transcriptional level. B7H3 was first cloned by Chapoval et al⁵ as a member of the B7 costimulatory family of proteins. Later it was determined to exist as a type I membrane protein with four instead of two Ig-like domains, and hence given the new name of 41g-B7H3 (see Table 2).⁶ In vitro 4Ig-B7H3 was more inhibitory than costimulatory for T-cell activation.⁶ B7H3 protein expression has been detected in gastric, NSCLC, neuroblasotma and many human tumor cell lines.^{5,7, 8} Human neuroblastoma tumors and cell lines expressing 4Ig-B7H3 may inhibit an NK-mediated immune response.⁷ B7H3 was found to be expressed on 59% of gastric carcinoma and 100% of gastric adenoma samples,⁹ and appears to correlate with better survival. In murine models^{10, 11} and human melanoma,¹² B7H3 appears to evince anti-tumor response. Murine B7H3 promotes acute and chronic allograft rejection.¹³ B7H3 probably plays a role in potentiating tumor immunosurveillance while the 4Ig-B7H3 exerts an inhibitory effect.⁴ It is of interest that 4Ig-B7H3 is the major isoform in most issues except brain and placenta.¹⁴ In the placenta B7H3 is a 110 kd double band and a 60 kd single band by western blot.¹⁵ It was most prominent on the extravillous trophoblast throughout gestation. B7H3 is also thought to play a role in bone formation.¹⁶

The identification of 4Ig-B7H3 as the antigen for 8H9 suggests that this glycoprotein is highly expressed among human solid tumors. The epitope that 8H9 recognizes appears to be restricted to tumors versus normal tissues. Based on the mRNA work published to date, one would have inevitably concluded that this antigen is ubiquitous and unsuitable to be a tumor target. We, however, found otherwise. We postulate that antibody directed at 4Ig-B7H3 can be safely administered without major side effects as was seen recently with anti-CD28 antibodies or with anti-CTLA4 where T-cell are targeted. We believe that 4Ig-B7H3 is an immune coinhibitory molecule, and antibodies like 8H9 can modulate its function and potentiate host anti-tumor immune response across a spectrum of human cancers.

### Example 4

### Isolation and Identification of B7H3 Receptors on Activated NK/T Cells Using 8H9 Monoclonal Antibodies

Monoclonal antibody 8H9 recognizes the 4Ig domain isoform of the human B7-homolog 3, 4Ig-B7H3. Human B7-homolog 3 (B7H3), which is also known as CD276, is a molecule which is believed to provide negative signals to the immune system, in particular providing negative signals to NK/T cells, allowing tumor cells to escape immune response. The identified antigen 4Ig-B7H3 targeted by monoclonal antibody 8H9 is the dominant variant form of B7H3 (CD276). 4Ig-B7H3 is a dominantly expressed form of human B7H3 containing a splice variation that duplicates the V-like and C-like Ig domain.^{14, 6}

As an immune modulator, both positive and negative immunologic functions of B7H3 have been reported. Reports describing the 2Ig-B7H3 variant demonstrated that the role of B7H3 was to promote T cell activation and IFN-γ production by binding to a putative receptor on activated T cells.⁵ Antitumor response was enhanced by B7H3 expression in murine tumor models.¹¹ In patients, B7H3 positivity in gastric carcinoma was correlated with increased survival.⁹ Conversely, the coinhibitory role of B7H3 was supported by reports that both 2Ig-B7H3 and 4Ig-B7H3 inhibited T cell proliferation and cytokine production⁶, that B7H3 preferentially downregulated TH1-mediated immune response in B7H3-deficient mice¹⁷, and that 4Ig-B7H3 inhibited NK-mediated lysis of neuroblastoma cells by interacting with a putative inhibitory receptor in the surface of NK cells⁷. The contradictory findings were possibly explained by the antagonistic B7H3 receptors.

The following example describes how B7H3 receptors on activated NK/T cells may be identified and isolated. This experiment has not yet been performed.

B7H3 receptors on NK/T cells are purified by affinity chromatography using both 2Ig-B7H3-Fc and 4Ig-B7H3-Fc as the baits. A B7H3-Fc fusion protein is created in the following manner: 2Ig-B7H3-Fc is purchased from R & D Systems while the cDNA sequence encoding the extracellular domain of human 4Ig-B7H3 is fused to the Fc region of mouse IgG2a using the pFUSE-mlg-G2a-Fc2 expression vector. The fusion protein is expressed in the CG44-CHO cell line and purified by affinity chromatography using protein A Sepharose. Purity and functionality of the fusion protein are evaluated by coomassie blue staining and anti-B7H3 Western blot.

NK/T cells positive for the B7H3 receptor are selected for. The established NK/T cell lines NK92, NKL, NK3.3, YT, TALL-104, as well as activated NK/T cells enriched from fresh peripheral blood mononuclear cells (PBMC) are incubated with B7H3-Fc with subsequent staining with fluorescence-conjugated secondary antibody, and analyzed by fluorescence activated cell sorting (FACS). The positive cells are further confirmed by B7H3-Fc Western blot.

The NK/T cells selected as being positive for B7H3 receptors are used for affinity purification. A B7H3-Fc affinity column is prepared by covalently conjugating the Fc portion of B7H3-Fc to protein G on the gel matrix using Protein G IgG Plus Orientation Kit (Pierce Biotechnology). Cell extracts from B7H3 receptor-positive cells are incubated with the Sepharose beads on the column. The column is washed extensively and eluted. The presence and purity of B7H3 receptor is monitored by B7H3-Fc Western blot and silver staining. B7H3 receptor-positive bands of over 20 ng are sent for mass spectrometric identification.

### Example 5

### Use of the 8H9 Monoclonal Antibody for Blockage of Inhibitory B7H3 (CD276) and Subsequent Enhanced NK/T Cell-Mediated Cytolysis of Tumor Cells

Immune responses to tumors have been found to be enhanced by blocking inhibitory receptors on T cells with monoclonal antibodies specific to said inhibitory receptors. A known example of this phenomenon is the enhancement of immune response through the blockade of CTLA-4 inhibitory receptor on T cells using anti-CTLA-4 monoclonal antibodies. The following example describes how a blockade of B7H3 receptor on NK/T cells with 8H9 antibody sensitizes tumor cells to NK/T cell-mediated toxicity. This experiment has not yet been performed.

Cell-Mediated Cytolysis (Chromium release) Assay: For the NK cell-mediated cytolysis assay, human CML cell line K562 is chosen for the target cells. As demonstrated by FACS analysis, K562 has low expression of HLA-1 and B7H3 proteins. Rhabdomyosarcoma HTB82 cells are used as a control. In a standard 4 hour ⁵¹Cr-release assay, while only less than 10% of rhabdomyosarcoma HTB82 cells were lysed by NK92 cells, up to 60% of K562 cells were effectively killed by NK92 effector cells. One group of the target cell population of K562 is transfected with nucleic acids encoding for the splice forms 4Ig-B7H3 in order that B7H3 be overexpressed in this cell population. The K562 target cells are radiolabeled with 100 μCi ⁵¹Cr/10⁶ cells for 1 hour at 37° C. The monoclonal antibody 8H9 is incubated with the transfected target cells while controls are incubated with HLA-1 mAb HB95, and cytolysis assays are performed using effector cells positive for the coinhibitory B7H3 receptor. NK92 effector cells are incubated in 96-well plates with target cells in 250 μl for 4 hours at 37° C. Cytolytic activity of NK92 effector cells against B7H3 transfected K562 will be decreased vis-à-vis non-transfected K562. Restored cytolytic activities will be observed after blocking of the coinhibitory B7H3.

### REFERENCES

1. Modak S, Kramer K, Gultekin S H, et. al.: Monoclonal antibody 8H9 targets a novel cell surface antigen expressed by a wide spectrum of human solid tumors. Cancer Res. 61:4048-54, 2001.
2. Modak S, Gerald W, Cheung N K: Disialoganglioside GD2 and a novel tumor antigen: potential targets for immunotherapy of desmoplastic small round cell tumor. Med. Pediatr. Oncol. 39:547-51, 2002.
3. Modak S, Guo H F, Hum J L, et al: Radioimmunotargeting of human rhabdomyosarcoma using monoclonal antibody 8H9. Cancer Biother. Radiopharm. 20:534-46, 2005.
4. Flies D B, Chen L: The new B7s: playing a pivotal role in tumor immunity. J. Immunother. 30:251-60, 2007.
5. Chapoval A I, Ni J, Lau J S, et. al.: B7-H3: a costimulatory molecule for T cell activation and IFN-gamma production. Nat. Immunol. 2:269-74, 2001.
6. Steinberger P, Majdic O, Derdak S V, et. al.: Molecular characterization of human 4Ig-B7-H3, a member of the B7 family with four Ig-like domains. J. Immunol. 172:2352-9, 2004.
7. Castriconi R, Dondero A, Augugliaro R, et. al.: Identification of 4Ig-B7-H3 as a neuroblastoma-associated molecule that exerts a protective role from an NK cell-mediated lysis. Proc. Natl. Acad. Sci. USA 101:12640-5, 2004.
8. Sun Y, Wang Y, Zhao J, et. al.: B7-H3 and B7-H4 expression in non-small-cell lung cancer. Lung Cancer 53:143-51, 2006.
9. Wu C P, Jiang J T, Tan M, et. al.: Relationship between co-stimulatory molecule B7-H3 expression and gastric carcinoma histology and prognosis. World J. Gastroenterol. 12:457-9, 2006.
10. Luo L, Chapoval A I, Flies D B, et. al.: B7-H3 enhances tumor immunity in vivo by costimulating rapid clonal expansion of antigen-specific CD8+ cytolytic T cells. J. Immunol. 173:5445-50, 2004.
11. Sun X, Vale M, Leung E, et. al.: Mouse B7-H3 induces antitumor immunity. Gene Ther. 10:1728-34, 2003.
12. Lupu C M, Eisenbach C, Kuefner M A, et. al.: An orthotopic colon cancer model for studying the B7-H3 antitumor effect in vivo. J. Gastrointest. Surg. 10:635-45, 2006.
13. Wang L, Fraser C C, Kikly K, et. al.: B7-H3 promotes acute and chronic allograft rejection. Eur. J. Immunol. 35:428-38, 2005.
14. Sun M, Richards S, Prasad D V, et. al.: Characterization of mouse and human B7-H3 genes. J. Immunol. 168:6294-7, 2002.
15. Petroff M G, Kharatyan E, Torry D S, et. al.: The immunomodulatory proteins B7-DC, B7-H2, and B7-H3 are differentially expressed across gestation in the human placenta. Am. J. Pathol. 167:465-73, 2005.
16. Suh W K, Wang S X, Jheon A H, et. al.: The immune regulatory protein B7-H3 promotes osteoblast differentiation and bone mineralization. Proc. Natl. Acad. Sci. USA 101:12969-73, 2004.
17. Suh W K, Gajewska B U, Okada H, Gronski M A, Bertram E M, Dawicki W, Duncan G S, Bukczynski J, Plyte S, Elia A, Wakeham A, Itie A, Chung S, Da Costa J, Arya S, Horan T, Campbell P, Gaida K, Ohashi P S, Watts T H, Yoshinaga S K, Bray M R, Jordana M, Mak T W: The B7 family members B7H3 preferentially down-regulates T helper type 1-mediated immune responses. Nat. Immunol. 4: 899-906, 2003.

1-44. (canceled)

45. A method of improving the prognosis or prolonging the survival of a subject bearing a tumor, comprising administering to the subject a composition comprising an effective amount of an agent capable of binding to an antigen recognized by monoclonal antibody 8H9.

46. The method of claim 45, wherein the agent is a polypeptide comprising Complementary Determining Regions (CDRs) of monoclonal antibody 8H9.

47. The method of claim 46, wherein the CDRs have the sequences selected from the group consisting of SEQ ID NOs.1-6.

48. The method of claim 45, wherein the agent is a single chain antibody or an antibody-fusion construct.

49. The method of claim 45, wherein the antigen recognized by monoclonal antibody 8H9 is CD276.

50. The method of claim 45, wherein the agent is directly or indirectly coupled to a labeling agent or a cytotoxic agent.

51. The method of claim 50, wherein the cytotoxic agent or labeling agent is a radioisotope.

52. The method of claim 45, wherein the composition is administered after the subject has been treated with one or more other cancer treatments.

53. The method of claim 52, wherein the other cancer treatments are selected from the group consisting of surgery, chemotherapy, and radiation.

54. The method of claim 45, wherein the tumor expresses an antigen recognized by monoclonal antibody 8H9.

55. The method of claim 45, wherein the composition is administered by a method selected from the group consisting of intravenous injection, intrathecal injection, injection by Ommaya reservoir or by spinal tap, intraparenchymal injection into the tumor or into tissues surrounding the tumor, and intraperitoneal injection.

56. The method of claim 45, wherein the agent is administered at 0.01 mg to 20 mg per injection, carrying 1 mCi to 100 mCi of 13I-iodine, 124-iodine, or biologically equivalent radioactive dosages of beta-emitters, alpha emitters or positron emitters.

57. The method of claim 45, wherein the tumor is neuroblastoma or metastatic neuroblastoma.

58. A method of screening for antibodies that have the same or similar binding specificity as monoclonal antibody 8H9, comprising the step of contacting candidate antibodies with a polypeptide comprising the sequence of SEQ ID NO.15, or a fragment thereof, wherein antibodies that bind to the polypeptide are antibodies that have the same or similar binding specificity as monoclonal antibody 8H9.

59. An antibody identified by the method of claim 58.

60. A method of upregulating anti-metastatic immune response in NK or T cells comprising the steps of blocking B7H3 receptors present on NK or T cells with an appropriate agent.

61. The method of claim 60, wherein the agent is an antibody.

62. The method of claim 61, wherein the antibody is monoclonal antibody 8H9.
